(19) United States
(12) Patent Application Publication
(10) Pub. No.: US 2019/0254851 A1
(43) Pub. Date: Aug. 22, 2019

(54) Elbow Orthosis

(71) Applicants: Alexander Bryan Carlson, Lincoln, NE (US); Scott Edwin Strasburger, Lincoln, NE (US)

(72) Inventors: Alexander Bryan Carlson, Lincoln, NE (US); Scott Edwin Strasburger, Lincoln, NE (US)

(21) Appl. No.: 16/278,981

(22) Filed: Feb. 19, 2019

Related U.S. Application Data
(60) Provisional application No. 62/632,243, filed on Feb. 19, 2018.

(51) Int. Cl. A61F 5/01

(52) U.S. Cl. CPC A61F 5/013; A61F 2005/0172; A61F 2005/0158

(57) ABSTRACT

The present invention relates to the field of orthopedic braces, made for the elbow, that assists in restoring function and the structural characteristics of the associated joints, muscles, and tendons.

### BACKGROUND OF THE INVENTION

The elbow is one of the most heavily used joints, used in a variety of everyday activities. Injuries to the elbow can occur from overuse, incorrect repetitive mechanical motion, trauma associated with athletic participation, work, or accidents. These often require surgical repairs or reconstruction. An ulnar collateral ligament reconstruction (UCLR), also known as Tommy John surgery, is one such surgery that repairs a torn ulnar collateral ligament, located in the elbow. The ulnar collateral ligament (UCL), located on the inside of the elbow, connects the humerus bone in the upper arm to the ulna bone in the forearm. A UCL injury typically results from repetitive stress or trauma to the elbow. The UCL can develop small or large tears or can stretch and lengthen to the point where it no longer supports the elbow during throwing activities. During a UCLR, a tendon is taken from elsewhere in the patient's body, such as the wrist, forearm, or hamstring, and used to replace the weak, torn ligament. The surgeon then drills tunnels into the ulna and humerus and threads the tendon through the tunnels and securing them to the bone.

Post-surgical rehabilitation typically takes 12 to 18 months, sometimes longer, before the elbow returns to pre-injury form. The new “system” is very weak immediately after the surgery, and the rebuilding process must be gradual as the body needs adequate time to convert the inserted tendon, which attaches muscle to bone, into a ligament, which connects bone to bone. During recovery, the arm is fitted in a postoperative, range-of-motion orthosis used to protect the repair, restrict movement, and prevent valgus and varus stress.

Current postoperative elbow orthoses are limiting because they are cumbersome, difficult to adjust, only allow for static changes in positioning without the option for gradual range-of-motion extension, and do not provide the rehabilitative opportunity for forced hyperextension, making it difficult for the user to regain full extension of the elbow. Because the arm is often incapable of reaching full-end range-of-motion after surgery the disclosed orthosis offers an alternative solution by using the orthosis to passively force the arm into full extension for a more complete healing process.

A postoperative elbow orthosis is needed that provides therapeutic circumferential compression to the arm, allows for both sequential and dynamic extension, and provides gradual range-of-motion extension, allowing up to 10° of forced hyperextension.

### BRIEF DESCRIPTION OF THE DRAWINGS

FIG. 1 illustrates a perspective view of the lateral side of the elbow orthosis.

FIG. 2 illustrates an additional perspective view of the medial side of the elbow orthosis.

FIG. 3 illustrates an additional perspective view of the medial side of the elbow orthosis.

FIG. 4 illustrates an addition perspective view of the lateral side of the elbow orthosis.

FIG. 5 illustrates a perspective view of the lateral side of the elbow orthosis without the compression sleeve.

FIG. 6 illustrates a perspective view of the lateral side of the orthosis without the compression sleeve in a position of 90° of flexion.

FIG. 7 illustrates a perspective view of the cable-tensioning system of the elbow orthosis.

FIG. 8 illustrates an exploded view of the dial, dial head, cable, and release mechanism of the elbow orthosis.

FIG. 9 illustrates an exploded view of the dial, dial head, cable, and one-way cable-tensioning system of the elbow orthosis.

FIG. 11 illustrates an exploded view of the elbow pad and elbow guard of the elbow orthosis.

FIG. 12 illustrates a diagram showing the positions flexion and extension of the elbow.

FIG. 13 illustrates a perspective view of the compression sleeve worn independently from the frame.

### BRIEF SUMMARY OF THE INVENTION

In a preferred embodiment, a postoperative, range-of-motion elbow orthosis which provides rehabilitative support for patients in the form of dynamic range-of-motion extension for the treatment of ligamentous injuries and stable fractures of the elbow and distal humerus is disclosed.

The orthosis consists of an asymmetric, s-shaped upper frame that lies medially beneath the axilla and laterally at the humeral head of the shoulder as well as a lower frame that lies near the distal portion of the radius and ulna. Together the upper and lower frame is designed to follow the contour of the arm, allowing the frame to extend up to the axilla and the shoulder, ultimately creating a longer lever to minimize the force required to achieve maximum extension. Constructed with lightweight materials, such as thermoplastic polyurethane, nylon, plastic, aluminum, or carbon fiber, the frame is comfortable and compact, eliminating unnecessary weight for the user. The frame is secured to the user through a series of proximal and distal straps and clasps, helping to prevent frame migration by providing a secure and customized fit to the user.

A circumferential compression sleeve, made of an elastic material such as spandex, lined with a gripping element such as silicone, prevents migration. The circumferential compression sleeve extends from the wrist to the shoulder, provides cushioning for comfort during movement, reduces swelling of the affected area, absorbs moisture produced by the skin, and provides ventilation while allowing the wrist and hand to move freely. The circumferential compression sleeve is applied independently and worn beneath the frame.

A cable-tensioning system, consisting of a dial, tensioning cable, cable-tunneling system, and cable guides, are used to create a customized tensioning system which allows for sequential, dynamic, and gradual forced extension to the elbow, progressively adjusting the orthosis' position of extension, allowing up to 10° of hyperextension.

The dial, consisting of a dial head, spool, one-way cable-tensioning system, and a release mechanism, located on the distal portion of the frame near the forearm, is easily accessed by the opposite hand of the user. Turning the dial clockwise creates tension and results in extension.

The cable-tensioning system causes the elbow guard, made of a rigid material such as thermoplastic polyurethane, aluminum, carbon fiber, or various other forms of plastic, the elbow into extension. The elbow pad, located on the interior portion of the elbow guard and consisting of foam or gel, mimics the shape of the elbow guard and conforms to the elbow. Together these serve as the fulcrum for the cable-tensioning system and provide added protection to the elbow.

A medial and lateral range of motion hinge, located between the upper portion of the frame and the lower portion of the frame, allows the orthosis to be locked into specific degrees of flexion or extension, and 10° of hyperextension.

The completed orthosis will be available in a variety of sizes, as well as a custom-fitted option.

Indications for this elbow orthosis include treatment of: ligamentous injury distal bicep tendon repair, tricep tendon repair, collateral ligament reconstruction, ulnar collateral ligament repair, lateral ulnar collateral ligament repair, radial collateral ligament repair, stable fractures of the elbow or upper arm, distal humerus fracture, proximal radius fracture, radial head fracture, ulna fracture, chronic elbow injury, elbow hyperextension, range-of-motion control post-injury, tennis elbow release, among others.

### DETAILED DESCRIPTION OF THE INVENTION

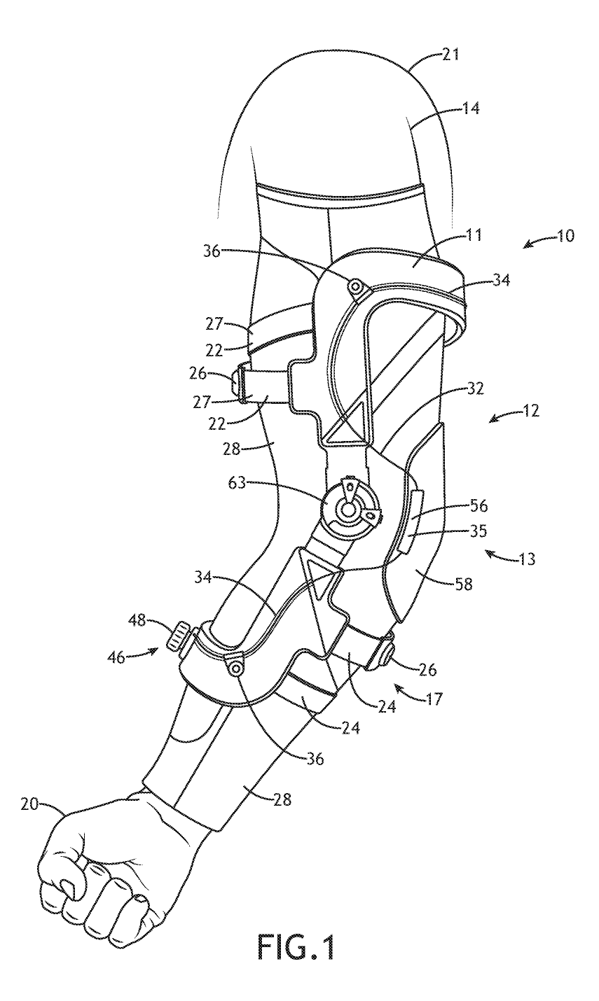

The present invention relates to an elbow orthosis 10, as shown in FIG. 1 which provides postoperative support that provides rehabilitative support to the elbow 13 in the form of sequential and dynamic extension, ultimately forcing the orthosis 10 into 10° or less of hyperextension 44, while controlling range-of-motion movement, and providing compression.

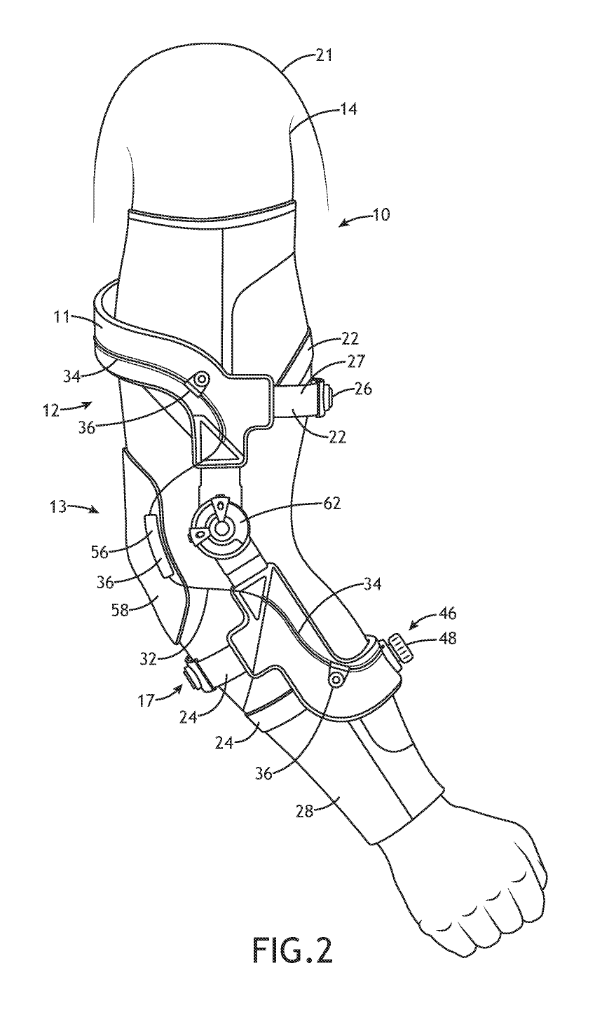

The orthosis 10 includes an asymmetric, s-shaped upper frame 12 that lies medially beneath the axilla 14 and laterally at the humeral head of the shoulder 21, as seen in FIG. 2. The orthosis 10 includes a lower frame 17 that lies near the distal portion of the radius and ulna providing support and protection to the affected area. Together the upper 12 and lower frame 17 is designed to follow the contour of the arm 18, allowing the frame 11 to extend up the arm 18 to the axilla 14, ultimately creating a longer lever to minimize the force required to achieve maximum extension. The frame 11, constructed with lightweight materials, such as thermoplastic polyurethane, nylon, aluminum, or carbon fiber, is comfortable and compact, eliminating unnecessary weight for the user 20. The frame 11 is secured to the user 20 through a series of proximal 22 and distal straps 24 and clasps 26, helping to prevent frame 11 migration by providing a secure and customized fit to the user 20. In one embodiment, the orthosis 10 includes one proximal bi-fabricated strap 27 and two distal straps 24.

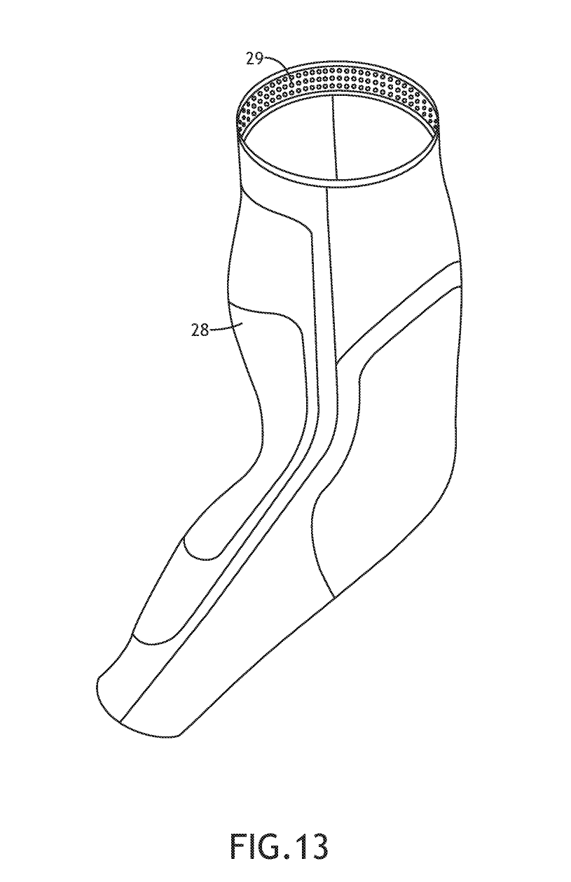

A circumferential compression sleeve 28, as seen in FIG. 13, made of an elastic material such as spandex, neoprene, or aeroprene lined with a gripping element 29, such as silicone, prevents migration. The circumferential compression sleeve 28, extending from the wrist 19 to the shoulder 21, provides cushioning for comfort during movement, reduces swelling of the affected area, absorbs moisture produced by the skin, and provides ventilation, while allowing the wrist 19 and hand 15 to move freely.

The circumferential sleeve 28 is applied independently and worn beneath the frame 11. In one embodiment, the compression sleeve 28 incorporates a closure system utilized to create a customized fit for the wearer 20. In another embodiment, the compression sleeve 28 provides post-rehabilitative compression and can be worn independently from the frame 11 during performance.

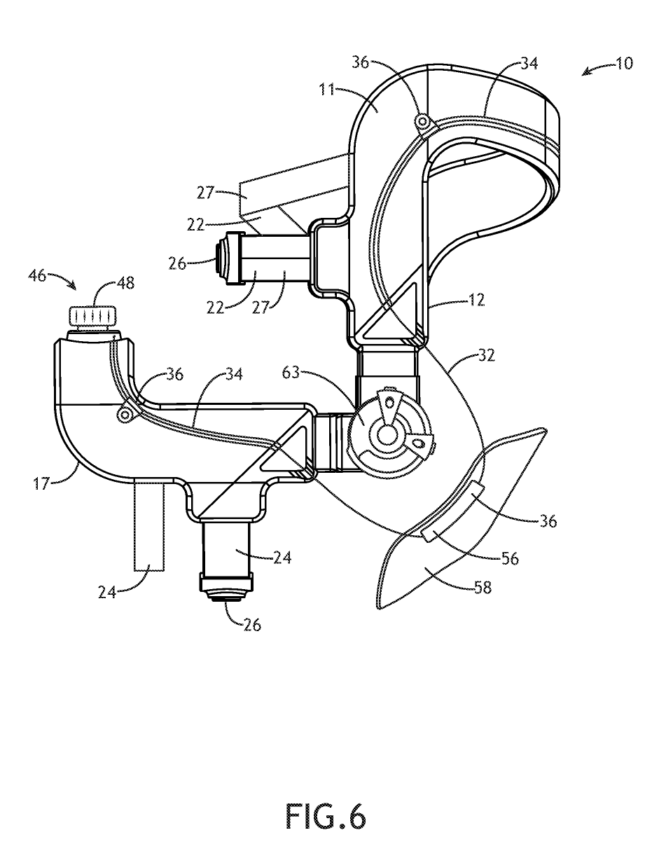

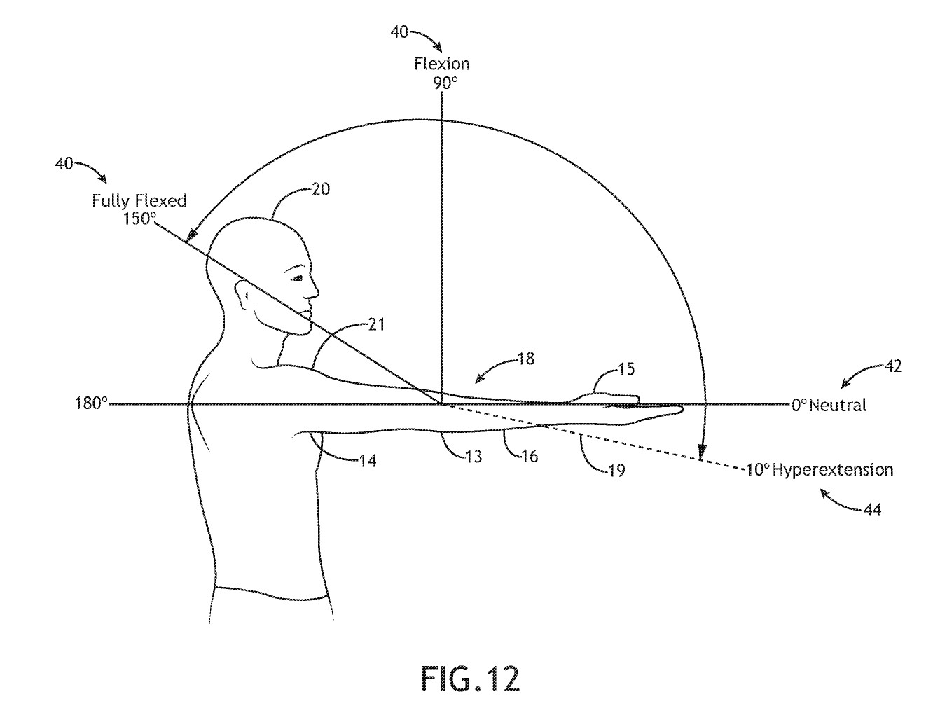

A cable-tensioning system 30, consisting of a dial 46, tensioning cable 32, cable-tunneling system 34 etched into the frame 11, cable guides 36, and a u-joint 56 adhered to the elbow guard 58, as shown in FIG. 6, are used to create a customized cable-tensioning system which allows for sequential, dynamic, and gradual forced extension to the elbow 13, progressively adjusting the orthosis' 10 position of extension, allowing up to 10° of hyperextension 44 as shown in FIG. 12.

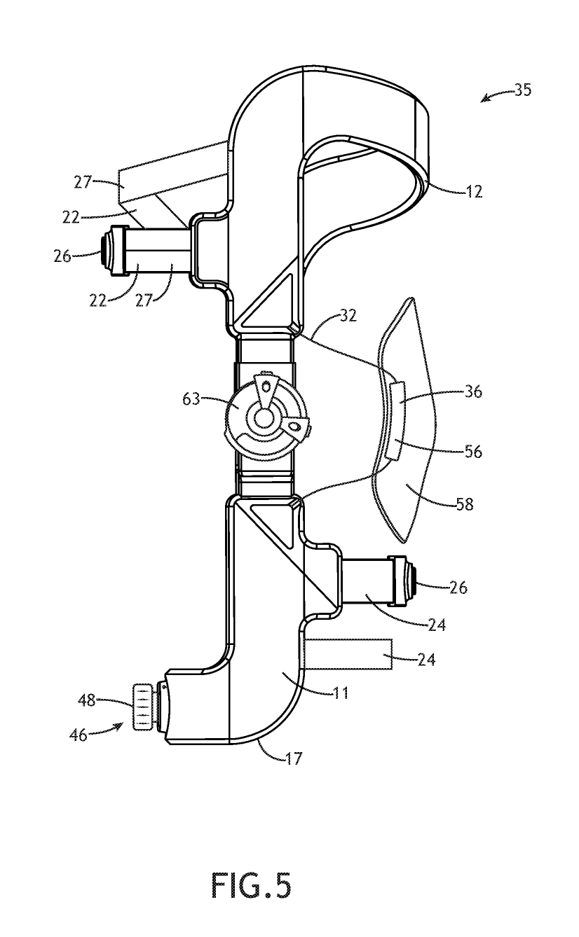

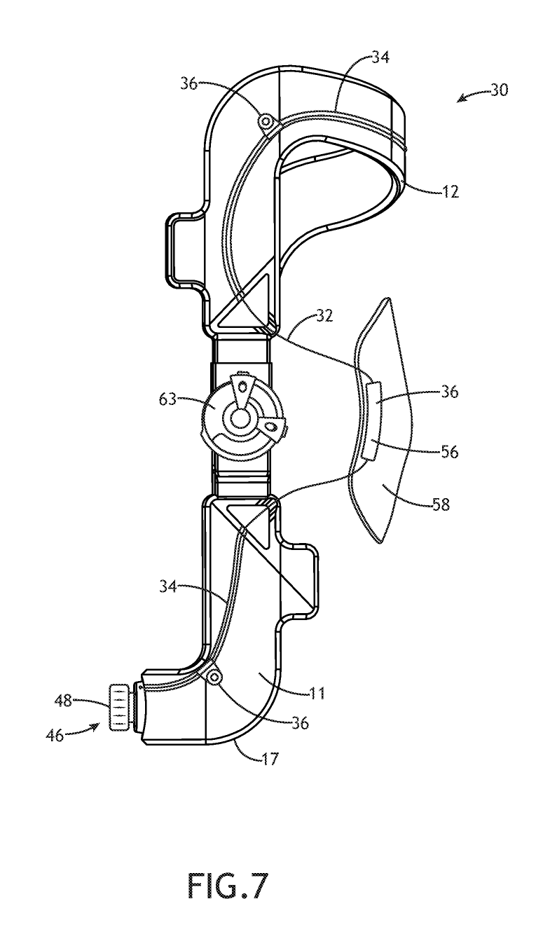

The tensioning cable 33 rests and moves along the frame 11 through cord guides 36 and a cable tunneling system 34. In one embodiment, the cable tunneling system 34 is integrated externally into the frame 11, as shown in FIG. 7. In another embodiment, the cable tunneling system 34 is integrated internally 35 into the frame 11, as shown in FIG. 5.

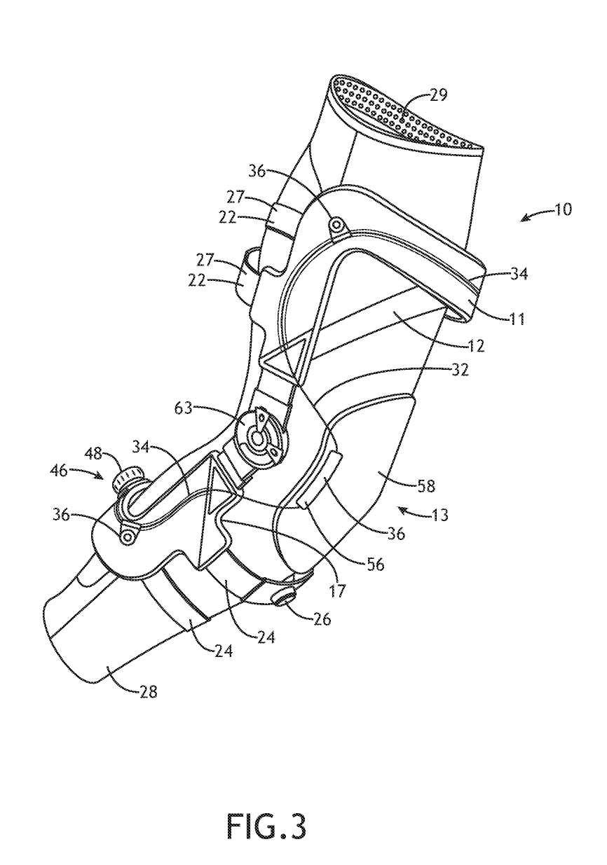

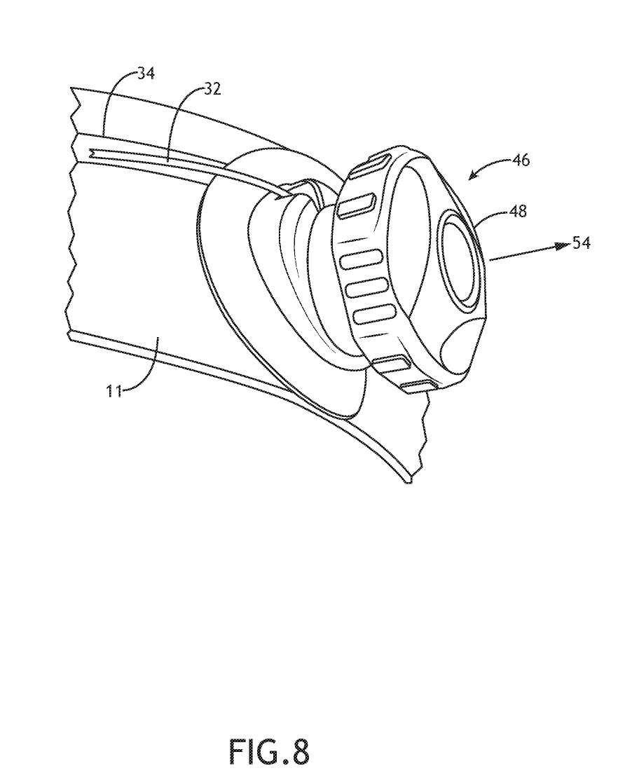

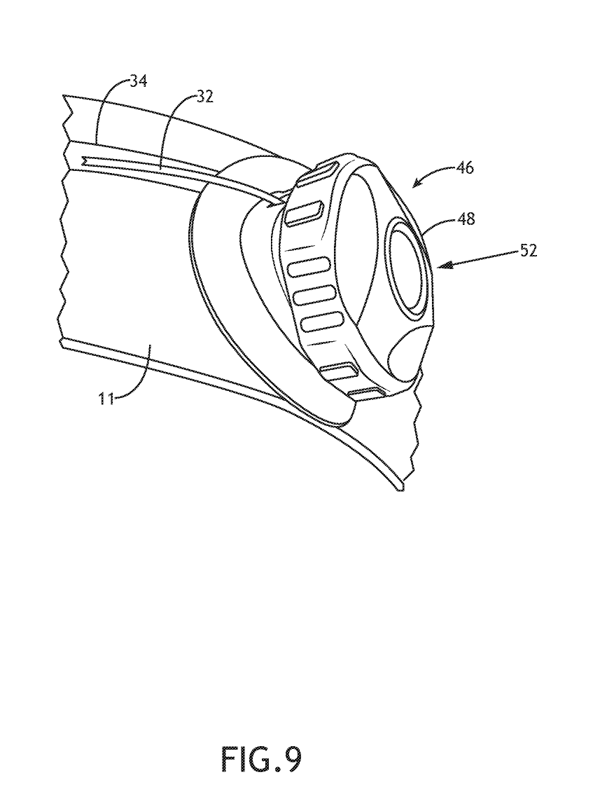

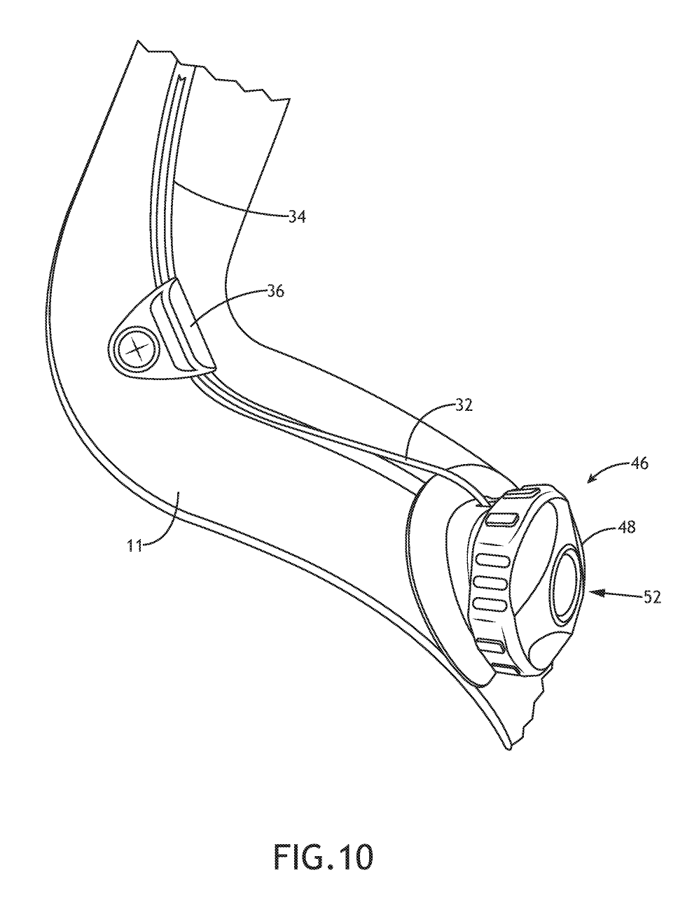

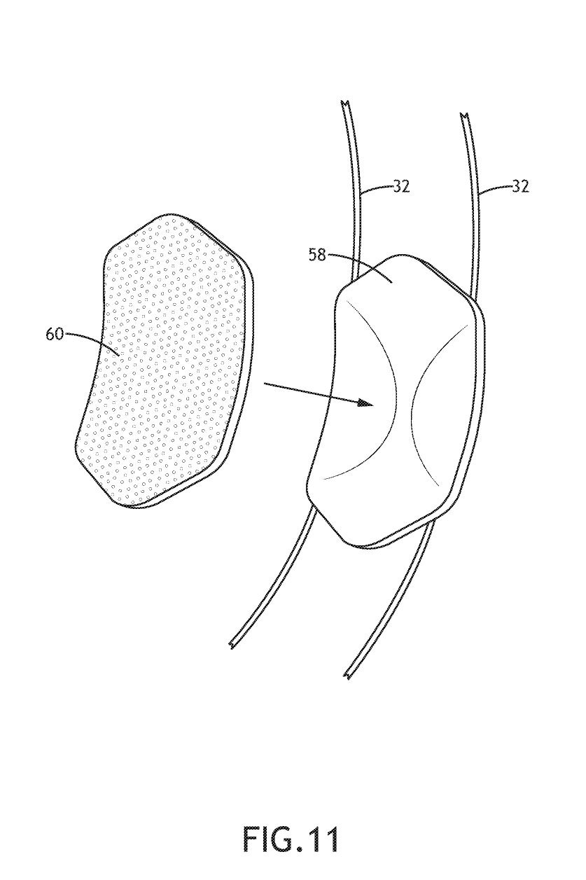

The dial 46, consisting of a dial head 48, spool 50, one-way cable-tensioning system 52, and a release mechanism 54, as shown in FIGS. 8,9, and 10, located on the distal lower frame 17 near the forearm 16, is easily accessed by the opposite hand of the user 20. Turning the dial 46 clockwise creates tension in the cable 32 and results in flexion 40. In another embodiment, the dial 56 is located on the bicep, or the anterior side of the forearm 16. The cable-tensioning system 30 causes the elbow guard 58, as shown in FIG. 11, made of a rigid material such as thermoplastic polyurethane, aluminum, carbon fiber, or various other forms of plastic, into extension 42. The elbow guard 58 hosts a u-joint cable guide 56 which provides a large surface area to help distribute the force applied by the cable-tensioning system 34, as shown in FIG. 3.

The elbow pad 60 located on the interior portion of the elbow guard 58, shown in FIG. 11, and consisting of foam or gel, mimics the shape of the elbow guard 58 and conforms to the elbow 13. Together these serve as the fulcrum for the cable-tensioning system 30 and provide added protection to the elbow 13.

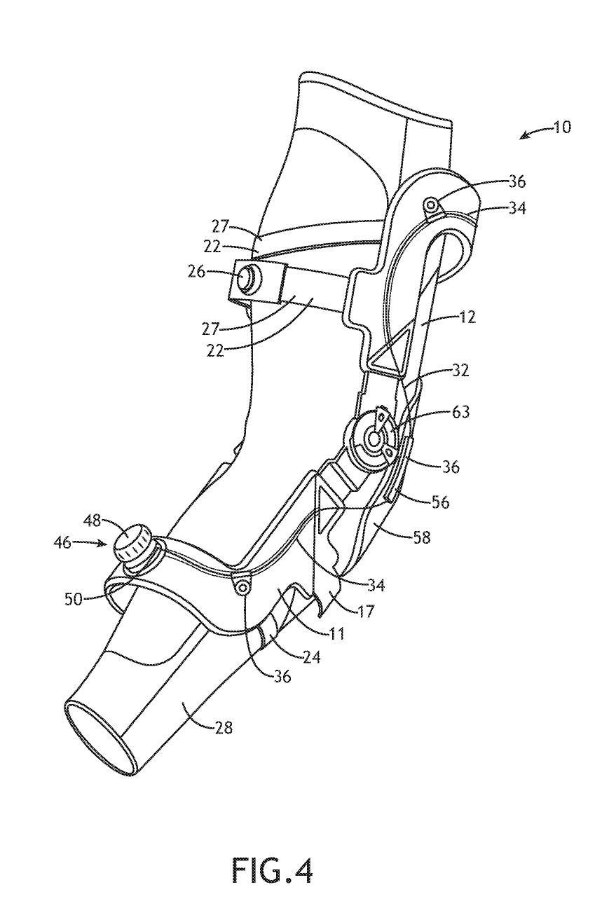

A medial 62 and lateral 63 range-of-motion hinge, as shown in FIGS. 2 and 4, located between the upper frame 12 and the lower frame 17, allows the orthosis 10 to be locked into specific degrees of flexion 40 or extension 42, and 10° of hyperextension 44.

The completed orthosis 10 will be available in a variety of sizes, as well as a custom-fitted option.

Indications for this elbow orthosis 10 include treatment of: ligamentous injury distal bicep tendon repair, tricep tendon repair, collateral ligament reconstruction, ulnar collateral ligament repair, lateral ulnar collateral ligament repair, radial collateral ligament repair, stable fractures of the elbow or upper arm, distal humerus fracture, proximal radius fracture, radial head fracture, ulna fracture, chronic elbow injury, elbow hyperextension, range-of-motion control post-injury, tennis elbow release, among others.

We claim:

1. An orthosis configured to support and treat the elbow comprising:
a circumferential compression sleeve;
an asymmetrical upper frame and a lower frame joined together medially and laterally by lockable range-of-motion hinges;
a series of proximal and distal straps;
an elbow guard,
an elbow pad, and
a one-way cable-tensioning system that consists of a dial, tensioning cable, cable tunneling system, cable guides, and a release mechanism.

2. An orthosis according to claim 1 where the elbow guard hosts a u-joint cable guide.

3. An orthosis according to claim 2 where the elbow guard is secured to the orthosis by the tensioning cable and the u-joint cable guide.

4. An orthosis according to claim 3 where the elbow guard is constructed with a rigid material.

5. An orthosis of claim 1 where the elbow pad mimics the shape of the elbow guard.

6. An orthosis of claim 5 where the elbow pad consists of cushion-like material.

7. An orthosis of claim 1 where the orthosis spans the length of the arm, from axilla and shoulder to wrist.

8. An orthosis of claim 1 where the compression sleeve is worn beneath the frame or independently of the frame.

9. An orthosis of claim 8 where the compression sleeve is lined with a gripping element.

10. An orthosis of claim 8 where the compression sleeve is constructed of stretch material.

11. An orthosis of claim 1 where the tunneling system is integrated internally or externally into the frame.

12. An orthosis of claim 1 where the frame is constructed of a rigid material.

13. An orthosis of claim 1 where the dial is located on the frame.

14. We claim a method of using an orthosis to achieve full range of motion of the elbow joint postoperatively comprising:
an arm-length, circumferential sleeve configured to reduce swelling;
a rigid frame, consisting of an asymmetrical upper frame and a lower frame, joined together medially and laterally by lockable range-of-motion hinges, a series of proximal and distal straps, an elbow guard, and an elbow pad, configured to provide support, adjustable range of motion, and produce a lever-like function to achieve maximum extension; and
a cable-tensioning system consisting of a dial, tensioning cable, cable tunneling system, cable guides, one-way cable-tensioning system, and a release mechanism configured to allow for sequential, dynamic, and gradual forced extension to the elbow, progressively adjusting the orthosis' position of extension, allowing up to 10° of hyperextension of the orthosis, ultimately achieving full extension of the elbow.

15. A method of claim 14 where the elbow guard hosts a u-joint cable guide which provides sufficient surface area to distribute the force applied by the cable-tensioning system.

16. A method of claim 14 where medial and lateral range-of-motion hinges, located between the upper frame and the lower frame, allows the orthosis to be locked into specific degrees of flexion or extension.

17. A method of claim 14 where the range-of-motion hinges are in an unlocked position, allowing the orthosis, together with the cable-tensioning system, to gradually force the orthosis into hyperextension.

18. A method of claim 14 where the elbow pad and elbow guard serve as the fulcrum for the cable-tensioning system and provide added protection to the elbow.

19. An orthosis configured to support and treat the elbow comprising:
a circumferential compression sleeve;
a two-piece frame joined by a hinge;
a series of straps;
an elbow guard, and
a cable-tensioning system.
